(19) United States
(12) Patent Application Publication
(10) Pub. No.: US 2025/0186384 A1
(43) Pub. Date: Jun. 12, 2025

(54) COMPOSITIONS COMPRISING 15-OHEPA AND METHODS OF USING THE SAME

(71) Applicant: Afimmune Limited, Dublin (IE)

(72) Inventors: Jonathan Rowe, Waterford, CT (US); Kevin Duffy, Dublin (IE); John Climax, Dublin (IE)

(21) Appl. No.: 18/531,259

(22) Filed: Dec. 6, 2023

Related U.S. Application Data
(63) Continuation of application No. 17/702,294, filed on Mar. 23, 2022, now abandoned.
Application No. 17/702,294 is a continuation of application No. 17/022,180, filed on Sep. 16, 2020, now abandoned.
Application No. 17/022,180 is a continuation of application No. 16/527,841, filed on Jul. 31, 2019, now Pat. No. 10,813,903.
Application No. 16/527,841 is a continuation of application No. 14/695,865, filed on Apr. 24, 2015, now abandoned.
Application No. 14/695,865 is a continuation of application No. PCT/EP2014/051455, filed on Jan. 24, 2014.

(30) Foreign Application Priority Data
Jan. 30, 2013 (GB) 1301626.6

(51) Int. Cl. A61K 31/202; A61K 9/48; A61P 1/16

(52) U.S. Cl. CPC A61K 31/202; A61K 9/48; A61P 1/16

(57) ABSTRACT

The present invention relates to the compositions, formulations and methods of treating fatty liver disorders, such as non-alcoholic fatty liver disease (NAFLD) and non-alcoholic steatohepatitis (NASH) and their sequelae by administration of 15-OHEPA.

### TECHNICAL FIELD

The invention pertains to the compositions, formulations and methods of treating fatty liver disorders (FLD), such as non-alcoholic fatty liver disease (NAFLD) and non-alcoholic steatohepatitis (NASH) and their sequelae by administration of a pharmaceutical composition comprising 15-hydroxy eicosapentaenoic acid (known as 15-OHEPA or 15-HEPE) in a subject in need thereof. In particular, the invention relates to a pharmaceutical composition with improved efficacy over one comprising EPA as the significant active component, rather than 15-OHEPA, for the treatment of subjects suffering from FLD and/or complications of FLD, to reduce fatty deposits in the liver to treat or prevent FLD and its associated complications.

### SUMMARY

Fatty liver disorders, also known as fatty liver or fatty liver disease (FLD), relates to a condition where large vacuoles of triglyceride fat accumulate in liver cells via the process of steatosis, or abnormal retention of lipids within a cell. Despite having multiple causes, fatty liver is considered a single disease that occurs frequently in subjects with excessive alcohol intake and/or those who are obese (with or without effects of insulin resistance). The condition is also associated with other diseases that influence fat metabolism. FLD may be categorized into two separate conditions: alcoholic FLD and non-alcoholic FLD. Both conditions show micro-vesicular and macro-vesicular fatty changes at different stages of the disease. Accumulation of fat may also be accompanied by a progressive inflammation of the liver (hepatitis), called steatohepatitis. Fatty liver is also known in the art as alcoholic steatosis and non-alcoholic fatty liver disease (NAFLD), and the more severe forms as alcoholic steatohepatitis (part of alcoholic liver disease) and non-alcoholic steatohepatitis (NASH). Nonalcoholic fatty liver disease-associated cirrhosis is the most severe form of the disease and is characterized by liver inflammation that leads to scarring of the liver tissue, ultimately resulting in liver failure.

Obesity, metabolic syndrome, type 2 diabetes, and atherosclerosis are increasing at an alarming rate in the Western world. In recent years, fatty liver has emerged as an independent risk factor for these diseases. Fatty liver is the accumulation of triglycerides and other fats within hepatocytes. Fatty liver disease can range from fatty liver alone (also known as “steatosis”), to fatty liver associated with inflammation or steatohepatitis. Non-alcoholic fatty liver disease (NAFLD) and non-alcoholic steatohepatitis (NASH) are the most common causes of chronic liver disease in the adult population and represents a crucial risk factor for progression to liver failure, cirrhosis and hepatocellular carcinoma. While steatosis affects approximately 30% of the population, 80% of obese patients have NAFLD and 50% of patients undergoing bariatric surgery have steatohepatitis. NAFLD also represents the most common cause of liver disease in children. It is estimated that NAFLD affects up to 20 percent of adults and nearly 5 percent of children. Some experts estimate that about two thirds of obese adults and half of obese children may have fatty liver. In the past ten years, as the rates of obesity in have doubled in adults and tripled in children and teenagers, NAFLD and NASH are becoming more common. NASH can cause scarring and hardening of the liver, leading to cirrhosis, a very serious disease that may require a liver transplant, and eventually to hepatocellular carcinoma.

There is no single established medical treatment for fatty liver. Presently, treatment of NAFLD is limited to 1) treatment of associated metabolic disorders such as diabetes and hyperlipidemia; 2) the management of insulin resistance focusing on weight loss, exercise and/or a pharmacological approach; and 3) the use of antioxidants as hepatic protection agents. Despite the use of many different therapeutic modalities, no clear treatment is currently available to address NAFLD. Because it is clinically important to resolve NAFLD and its sequelae, new approaches aimed at preventing and reversing fat accumulation in the liver are necessary.

We have surprisingly found that 15-HEPE, a metabolite of EPA, is more potent than EPA in the treatment of FLD.

### BRIEF DESCRIPTION OF THE DRAWINGS

FIGS. 1A, 1B, 2A, and 2B present data from an in vivo efficacy study of 15-OHEPA and EPA in STAM model of non-alcoholic steatohepatitis, as discussed in the Examples herein.

FIG. 1A shows that there was no significant change in body weight in any of the experimental groups. FIG. 1B shows that only the 15-OHEPA treatment group (500 mg/kg) and the positive control (telmisartan) had a significant reduction in liver weight as compared to the vehicle control.

FIGS. 2A and 2B show that NAS significantly decreased only in the 15-OHEPA (500 mg/kg) and the positive control (telmisartan) groups as compared to the vehicle control.

Other features and advantages of the invention will be apparent from the following detailed description.

### DETAILED DESCRIPTION

The present invention relates to compositions and methods for treating fatty liver disorders, including non-alcoholic fatty liver disease (NAFLD) and non-alcoholic steatohepatitis (NASH), by administration of a composition comprising 15-HEPE in a subject in need thereof.

As used herein, “15-OHEPA” is 15-Hydroxy-eicosa-5,8,11,13,17-pentaenoic acid. 15-OHEPA can be synthesized from eicosapentaenoic acid, EPA according to methods known in the art. As used herein, the term “15-OHEPA” refers to 15-OHEPA in its free acid form (e.g, 15-hydroxy-eicosa-5,8,11,13,17-pentaenoic acid) and/or a pharmaceutically acceptable ester, conjugate or salt thereof, or mixtures of any of the foregoing. A derivative of 15-OHEPA may be used instead, though this does not include any derivative compound missing the hydroxy group of 15-OHEPA. In some embodiments, the 15-OHEPA is used in the free acid form. Alternatively, pharmaceutically acceptable esters or salts of 15-OHEPA are used in the invention. In some embodiments, the 15-OHEPA is in the form of a C₁₋₄ alkyl ester such as methyl ester or ethyl ester form.

As used herein, “EPA” is eicosa-5,8,11,14,17-pentaenoic acid, also known as 20:5n-3, an omega-3 fatty acid. EPA is readily obtainable through commercial sources.

Accordingly, in one aspect of the present invention, a method of treating a fatty liver disorder in a subject is provided, comprising administering to the subject a therapeutically effective amount of a composition comprising 15-OHEPA.

The present invention provides 15-OHEPA, or a composition comprising 15-OHEPA, for use in the treatment of a fatty liver disorder.

The present invention provides a use of 15-OHEPA, or a composition comprising 15-OHEPA, in the manufacture of a medicament for treating a fatty liver disorder.

In another aspect, the present invention provides a pharmaceutical composition comprising a therapeutically effective amount of 15-OHEPA. The 15-OHEPA may be the sole significant active ingredient in that composition and in the methods and uses as stated herein. The 15-OHEPA may be the sole active ingredient. Alternatively, the 15-OHEPA may be combined for co-formulation or co-administration with other agents for treating FLD. If an additional active agent is to be used, the 15-OHEPA can be co-formulated as a single dosage unit or can be formulated as two to a plurality of dosage units for coordinated, combination or concomitant administration.

The invention also provides formulations of 15-OHEPA and formulations comprising 15-OHEPA and methods of using these formulations for treating fatty liver disorders, including non-alcoholic fatty liver disease (NAFLD) and non-alcoholic steatohepatitis (NASH).

15-OHEPA is a chiral molecule and may be used in the(S)- or (R)-enantiomeric form, or as a racemic mixture. Used herein, “15-OHEPA” includes all such forms, with no limitation as to stereospecifcity. In another embodiment, the 15-OHEPA comprises the(S) form: 15(S)-Hydroxy-(5Z,8Z,11Z,13E,17Z)-eicosapentaenoic acid. In some embodiments, the 15-OHEPA may be used in the form of the ethyl ester. In other embodiments the 15-OHEPA may be used as the free acid.

The present invention further provides an pharmaceutical composition for oral delivery, comprising 15-OHEPA. That composition may comprise a pharmaceutically acceptable excipient. The 15-OHEPA may be in any form as discussed herein. The 15-OHEPA may be present from about 50 mg to about 3000 mg.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

### Pharmaceutical Compositions

While the present invention is capable of being embodied in various forms, the description below of several embodiments is made with the understanding that the present disclosure is to be considered as an exemplification of the invention, and is not intended to limit the invention to the specific embodiments illustrated. Headings are provided for convenience only and are not to be construed to limit the invention in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” In this manner, slight variations from a stated value can be used to achieve substantially the same results as the stated value. Also, the disclosure of ranges is intended as a continuous range including every value between the minimum and maximum values recited as well as any ranges that can be formed by such values. Also disclosed herein are any and all ratios (and ranges of any such ratios) that can be formed by dividing a recited numeric value into any other recited numeric value. Accordingly, the skilled person will appreciate that many such ratios, ranges, and ranges of ratios can be unambiguously derived from the numerical values presented herein and in all instances such ratios, ranges, and ranges of ratios represent various embodiments of the present invention.

### 15-Hydroxy Eicosapentaenoic Acid

In one embodiment, compositions of the invention comprise 15-OHEPA as an active ingredient. 15-OHEPA is the abbreviation for 15-Hydroxy eicosapentaenoic acid, a metabolite of eicosapentaenoic acid (EPA) that can be synthesized via ways known in the art, such as exposure of eicospentaenoic acid to the enzyme 15-lipoxygenase. As used herein, the term “15-OHEPA” refers to 15-OHEPA in its free acid form (e.g., 15-Hydroxy eicosapentaenoic acid) and/or a pharmaceutically acceptable ester, conjugate or salt thereof, or mixtures of any of the foregoing. A derivative of 15-OHEPA may be used instead, though this does not include any derivative compound missing the hydroxy group of 15-OHEPA. The term “pharmaceutically acceptable” in the present context means that the substance in question does not produce unacceptable toxicity to the subject or interaction with other components of the composition.

In one embodiment, the 15-OHEPA is in the form of an ester (also referred to herein as E-15-OHEPA or ethyl-15-OHEPA). In another embodiment, the 15-OHEPA comprises a C₁-C₅ alkyl ester of 15-OHEPA. In another embodiment, the 15-OHEPA comprises 15-OHEPA methyl ester, 15-OHEPA propyl ester, or 15-OHEPA butyl ester. In still another embodiment, the 15-OHEPA comprises the optically active 15 (S)-Hydroxy-(5Z,8Z,11Z,13E,17Z)-eicosapentaenoic acid. This isomer may be used in any of the forms discussed above.

In another embodiment, the 15-OHEPA comprises lithium 15-OHEPA, mono, di- or triglyceride 15-OHEPA or any other ester or salt of 15-OHEPA, or the free acid form of 15-OHEPA.

In various embodiments, the invention provides pharmaceutical compositions, for example orally deliverable compositions, comprising 15-OHEPA. In one embodiment, the compositions comprise a therapeutically effective amount of 15-OHEPA. In one embodiment, the pharmaceutical composition comprises about 0.1% to about 99%, about 1% to about 95%, about 5% to about 90% by weight of 15-OHEPA.

In one embodiment, the pharmaceutical composition comprises about at least about 70%, at least about 80% or at least about 90%, by weight, of 15-OHEPA. In one embodiment, the pharmaceutical composition comprises at least about 50%, at least about 60%, at least about 70%, at least about 80% or at least about 90%, by weight of 15-OHEPA.

In another embodiment, 15-OHEPA is present in a composition of the invention in an amount of about 1 mg to about 10,000 mg, 25 mg to about 7500 mg, about 25 mg to about 5000 mg, about 50 mg to about 5000 mg, about 50 mg to about 3000 mg, about 75 mg to about 2500 mg, or about 100 mg to about 1000 mg, for example about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1025 mg, about 1050 mg, about 1075 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, or about 2500 mg.

In one embodiment, 15-OHEPA present in a composition of the invention comprises at least 90% by weight 15-OHEPA (as the term “15-OHEPA” is defined and exemplified herein). 15-OHEPA compositions can comprise even higher purity 15-OHEPA, for example at least 95% by weight 15-OHEPA or at least 97% by weight 15-OHEPA, wherein the 15-OHEPA is any form of 15-OHEPA as set forth herein. The purity of 15-OHEPA can further be defined (e.g. impurity profile) by any of the descriptions of 15-OHEPA provided herein.

Above are discussed the amounts of the 15-OHEPA in the pharmaceutical composition and their purity. The nature of the essential fatty acids and their synthesis is such that the 15-OHEPA composition may include moieties from other essential fatty acids in the essential fatty acid metabolic cascade.

In one embodiment, a composition of the invention contains not more than about 10%, not more than about 9%, not more than about 8%, not more than about 7%, not more than about 6%, not more than about 5%, not more than about 4%, not more than about 3%, not more than about 2%, not more than about 1%, or not more than about 0.5%, by weight of other omega-3 fatty acids including alpha linolenic acid, stearidonic acid, docosahexaenoic acid (DHA) or derivatives thereof. In other embodiments there is substantially no, or no such other omega-3 fatty acids present.

In another embodiment, 15-OHEPA represents at least about 60%, at least about 70%, at least about 80%, at least about 90%, at least about 95%, at least about 97%, at least about 98%, at least about 99%, or 100%, by weight, of all fatty acids present in a composition of the invention.

There may be present some residual eicosapentaenoic acid from the synthesis of the 15-OHEPA. There may be not more than about 10%, not more than about 9%, not more than about 8%, not more than about 7%, not more than about 6%, not more than about 5%, not more than about 4%, not more than about 3%, not more than about 2%, not more than about 1%, or not more than about 0.5%, by weight EPA. Alternatively, there is substantially no, or no, EPA in a form which has not been modified to the hydroxyl-form.

### Additional Active Agents

In one embodiment, the pharmaceutical composition further comprises one or more additional active agent(s). In one embodiment, the pharmaceutical composition comprises an amount of the additional active agent that is less than the generally recognized therapeutically effective amount for that agent. In one embodiment, the pharmaceutical composition comprises an amount of the additional active agent that is equal to or greater than the generally recognized therapeutically effective amount for that agent.

EPA itself has beneficial properties in treating FLD and it is possible to combine the 15-OHEPA with EPA in an alternative embodiment.

In one embodiment, 15-OHEPA and one or more active agent(s) are present in a composition of the invention, or are co-administered in a weight ratio of 15-OHEPA: additional agent of about 1:1000 to about 1000:1, about 1:500 to about 500:1, about 1:100 to about 100:1, about 1:50 to about 50:1, about 1:25 to about 25:1, about 1:10 to about 10:1, about 1:5 to about 5:1, about 1:4 to about 4:1 about 1:3 to about 3:1, about 1:2 to about 2:1 or about 1:1.

### Dosage Forms

A composition for use in accordance with the disclosure can be formulated as one or more dosage units. The terms “dose unit” and “dosage unit” herein refer to a portion of a pharmaceutical composition that contains an amount of a therapeutic agent suitable for a single administration to provide a therapeutic effect. Such dosage units may be administered one to a plurality (i.e. 1 to about 10, 1 to 8, 1 to 6, 1 to 4 or 1 to 2) of times per day, or as many times as needed to elicit a therapeutic response.

In some embodiments, compositions of the invention are in the form of orally deliverable dosage forms or units. Non-limiting examples of suitable dosage forms include tablets (e.g. suspension tablets, bite suspension tablets, rapid dispersion tablets, chewable tablets, etc), caplets, capsules (e.g. a soft or a hard gelatin capsule or HPMC capsule), lozenges, sachets, cachets, troches, pellets, suspension, elixirs, syrups or any other solid dosage form reasonably adapted for oral administration. The terms “oral delivery” and “oral administration” herein include any form of delivery wherein the agent or composition is placed in the mouth of the subject under treatment, whether swallowed or not. This therefore includes buccal and sublingual administration, as well as esophagael administration.

Alternatively, compositions of the invention can also be formulated for rectal, topical, or parenteral (e.g. subcutaneous, intramuscular, intravenous and intradermal or infusion) delivery.

In discussing the amount of 15-OHEPA in a composition of the invention, this may be split over several dosage forms. There is a limit as to the size for oral administration. If a subject is to be administered 1 to 4 g 15-OHEPA a day, this may be by up to 4 capsules, each providing 1 g 15-OHEPA.

Compositions of the invention can be in the form of liquid dosage forms or dose units to be imbibed directly or they can be mixed with food or beverage prior to ingestion. Non-limiting examples of suitable liquid dosage forms include solutions, suspensions, elixirs, syrups, liquid aerosol formulations, and the like.

In another embodiment, compositions of the invention comprise one or more pharmaceutically acceptable excipients. The term “pharmaceutically acceptable excipient” herein means any substance, not itself a therapeutic agent, used as a carrier or vehicle for delivery of a therapeutic agent to a subject or added to a pharmaceutical composition to improve its handling or storage properties or to permit or facilitate formation of a unit dose of the composition, and that does not produce unacceptable toxicity or interaction with other components in the composition. By way of example only, a pharmaceutical composition according to the present disclosure may comprise one or more of: antioxidants, surfactants, preservatives, flavouring agents, co-solvents, viscosity aids, suspension aids, and lipophilic phases.

In one embodiment, the pharmaceutical composition comprises one or more antioxidants such as ascorbic acid, palmitic acid, ascorbyl palmitate, α-tocopherol, idebenone, ubiquinone, ferulic acid, coenzyme Q10, lycopene, green tea, catechins, epigallocatechin 3-gallate (EGCG), green tea polyphenols (GTP), silymarin, coffeeberry, resveratrol, grape seed, pomegranate extracts, genisten, pycnogenol, niacinamide, and the like. In one embodiment, the pharmaceutical composition comprises about 0.01 wt. % to about 2 wt. % of an antioxidant, for example about 0.01 wt. %, about 0.02 wt. %, about 0.03 wt. %, about 0.04 wt. %, about 0.05 wt. %, about 0.06 wt. %, about 0.07 wt. %, about 0.08 wt. %, about 0.09 wt. %, about 0.1 wt. %, about 0.11 wt. %, about 0.12 wt. %, about 0.13 wt. %, about 0.14 wt. %, about 0.15 wt. %, about 0.16 wt. %, about 0.17 wt. %, about 0.18 wt. %, about 0.19 wt. %, about 0.2 wt. %, about 0.21 wt. %, about 0.22 wt. %, about 0.23 wt. %, about 0.24 wt. %, about 0.25 wt. %, about 0.26 wt. %, about 0.27 wt. %, about 0.28 wt. %, about 0.29 wt. %, about 0.3 wt. %, about 0.31 wt. %, about 0.32 wt. %, about 0.33 wt. %, about 0.34 wt. %, about 0.35 wt. %, about 0.36 wt. %, about 0.37 wt. %, about 0.38 wt. %, about 0.39 wt. %, about 0.4 wt. %, about 0.41 wt. %, about 0.42 wt. %, about 0.43 wt. %, about 0.44 wt. %, about 0.45 wt. %, about 0.46 wt. %, about 0.47 wt. %, about 0.48 wt. %, about 0.49 wt. %, about 0.5 wt. %, about 0.51 wt. %, about 0.52 wt. %, about 0.53 wt. %, about 0.54 wt. %, about 0.55 wt. %, about 0.56 wt. %, about 0.57 wt. %, about 0.58 wt. %, about 0.59 wt. %, about 0.6 wt. %, about 0.61 wt. %, about 0.62 wt. %, about 0.63 wt. %, about 0.64 wt. %, about 0.65 wt. %, about 0.66 wt. %, about 0.67 wt. %, about 0.68 wt. %, about 0.69 wt. %, about 0.7 wt. %, about 0.71 wt. %, about 0.72 wt. %, about 0.73 wt. %, about 0.74 wt. %, about 0.75 wt. %, about 0.76 wt. %, about 0.77 wt. %, about 0.78 wt. %, about 0.79 wt. %, about 0.8 wt. %, about 0.81 wt. %, about 0.82 wt. %, about 0.83 wt. %, about 0.84 wt. %, about 0.85 wt. %, about 0.86 wt. %, about 0.87 wt. %, about 0.88 wt. %, about 0.89 wt. %, about 0.9 wt. %, about 0.91 wt. %, about 0.92 wt. %, about 0.93 wt. %, about 0.94 wt. %, about 0.95 wt. %, about 0.96 wt. %, about 0.97 wt. %, about 0.98 wt. %, about 0.99 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, or about 2 wt. % of the one or more antioxidant.

### Therapeutic Methods

The compositions and formulations disclosed herein may be used in the treatment of fatty liver disease. In one embodiment the fatty liver disease is non-alcoholic fatty liver disease. In another embodiment the fatty liver disease is non-alcoholic steatohepatitis. In one embodiment, the method comprises administering a pharmaceutical composition as disclosed herein to a subject once per day, twice per day, three times per day, or more than three times per day.

As used herein, “treating” or “treatment” of a disease, disorder, or condition includes at least partially: (1) preventing the disease, disorder, or condition, i.e. causing the clinical symptoms of the disease, disorder, or condition not to develop in a mammal that is exposed to or predisposed to the disease, disorder, or condition but does not yet experience or display symptoms of the disease, disorder, or condition; (2) inhibiting the disease, disorder, or condition, i.e., arresting or reducing the development of the disease, disorder, or condition or its clinical symptoms; or (3) relieving the disease, disorder, or condition, i.e., causing regression of the disease, disorder, or condition or its clinical symptoms. The term “prevention” in relation to a given disease or disorder means: preventing the onset of disease development if none had occurred, preventing the disease or disorder from occurring in a subject that may be predisposed to the disorder or disease but has not yet been diagnosed as having the disorder or disease, and/or preventing further disease/disorder development if already present.

An “effective amount,” as used herein, refers to the amount of an active composition that is required to confer a therapeutic effect on the subject. A “therapeutically effective amount,” as used herein, refers to a sufficient amount of an agent or a compound being administered which will relieve to some extent one or more of the symptoms of the disease, disorder, or condition being treated. In some embodiments, the result is a reduction and/or alleviation of the signs, symptoms, or causes of a disease, or any other desired alteration of a biological system. For example, in some embodiments, an “effective amount” for therapeutic uses is the amount of the composition including a compound as disclosed herein required to provide a clinically significant decrease in disease symptoms without undue adverse side effects. In some embodiments, an appropriate “effective amount” in any individual case is determined using techniques, such as a dose escalation study. The term “therapeutically effective amount” includes, for example, a prophylactically effective amount. In other embodiments, an “effective amount” of a compound disclosed herein, such as a compound of Formula (A) or Formula (I), is an amount effective to achieve a desired pharmacologic effect or therapeutic improvement without undue adverse side effects. In other embodiments, it is understood that “an effect amount” or “a therapeutically effective amount” varies from subject to subject, due to variation in metabolism, age, weight, general condition of the subject, the condition being treated, the severity of the condition being treated, and the judgment of the prescribing physician. The term “pharmaceutically acceptable” in the present context means that the substance in question does not produce unacceptable toxicity to the subject or interaction with other components of the composition.

Without further description, it is believed that one of ordinary skill in the art may, using the preceding description and the following illustrative examples, make and utilize the agents of the present disclosure and practice the claimed methods. The following working examples are provided to facilitate the practice of the present disclosure, and are not to be construed as limiting in any way the remainder of the disclosure.

### EXAMPLES

The purpose of this study was to examine the effects of 15-OHEPA and EPA in the STAM model of Non-alcoholic Steatohepatitis.

### Protocol

Pathogen-free15-day-pregnant C57BL/6 mice were obtained from Charles River Laboratories in Japan Inc. (Kanagawa, Japan). NASH was established in male mice by a single subcutaneous injection of streptozotocin (STZ) (Sigma, USA) after birth and feeding with a high fat diet (HFD; CLEA) Japan, Japan) ad libitum after 4 weeks of age (day 28±2). Mice were randomized into 5 groups of 8 mice at 5 weeks of age (day 35±2) the day before the start of treatment.

During the treatment period, individual body weight was measured daily as well as clinical signs of behavior and survival.

### Groups:

Group 1 (Vehicle): Eight NASH mice were orally administered vehicle [olive oil] in a volume of 10 mL/kg once daily from 5 to 9 weeks of age.

Group 2 (15-OHEPA 50 mg/kg): Eight NASH mice were orally administered vehicle supplemented with 15-OHEPA at a dose of 50 mg/kg once daily from 5 to 9 weeks of age.

Group 3 (15-OHEPA 500 mg/kg): Eight NASH mice were orally administered vehicle supplemented with 15-OHEPA at a dose of 500 mg/kg once daily from 5 to 9 weeks of age.

Group 4 (EPA 500 mg/kg): Eight NASH mice were orally administered vehicle supplemented with EPA at a dose 500 mg/kg once daily from 5 to 9 weeks of age.

Group 5 (positive control): Eight NASH mice were orally administered pure water supplemented with Telmisartan at a dose of 10 mg/kg once daily from 5 to 9 weeks of age.

At 9 weeks of age, all mice were sacrificed and the following data was recorded;
Individual liver weight
Liver to body weight ratio

Histopathological analyses of liver sections were performed using HE staining (to estimate NAFLD Activity score). For HE staining, sections were cut from paraffin blocks of liver tissue prefixed in Bouin's solution and stained with Lillie-Mayer's Hematoxylin (Muto Pure Chemicals, Japan) and eosin solution (Wako Pure Chemical Industries). NAFLD Activity score (NAS) was calculated according to the criteria of Kleiner (Kleiner DE. et al., Hepatology, 2005; 41:1313).

Statistical tests were performed using Bonferroni Multiple Comparison Test. P values <0.05 were considered statistically significant.

### Results

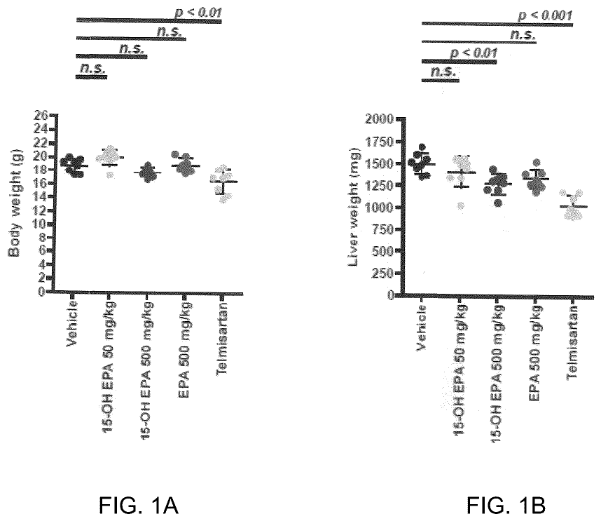

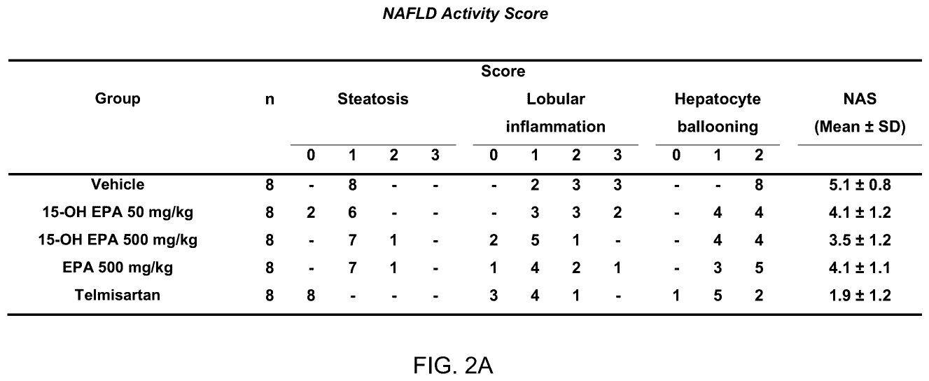

FIG. 1A shows that there was no significant change in body weight in any of the experimental groups.

FIG. 1B shows that only the 15-OHEPA treatment group (500 mg/kg) and the positive control (telmisartan) had a significant reduction in liver weight as compared to the vehicle control.

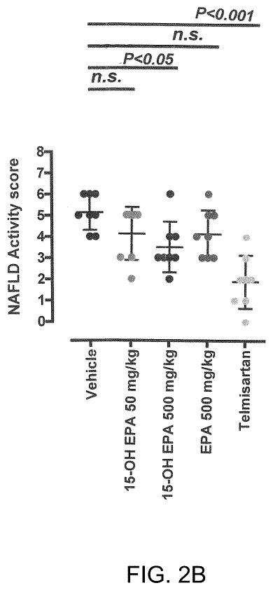

FIGS. 2A and 2B show that NAS significantly decreased only in the 15-OHEPA (500 mg/kg) and the positive control (telmisartan) groups as compared to the vehicle control.

1. An orally deliverable pharmaceutical composition comprising about 100 mg to 5000 mg of 15-hydroxyeicosapentaenoic acid (15-HEPE) in free form or a pharmaceutically acceptable ester, conjugate, or salt thereof enclosed in a capsule shell, wherein the 15-HEPE or pharmaceutically acceptable ester, conjugate or salt thereof is the sole active ingredient and comprises at least about 80%, by weight, of all fatty acids present in the orally deliverable pharmaceutical composition.

2. The orally deliverable pharmaceutical composition of claim 1, wherein the 15-HEPE is in esterified form.

3. The orally deliverable pharmaceutical composition of claim 2, wherein the esterified form is a C1-C5 alkyl ester.

4. The orally deliverable pharmaceutical composition of claim 2, wherein the esterified form is an ethyl ester.

5. The orally deliverable pharmaceutical composition of claim 2, wherein the esterified form is a triglyceride.

6. The orally deliverable pharmaceutical composition of claim 1, wherein the 15-HEPE is in salt form.

7. The orally deliverable pharmaceutical composition of claim 1, wherein an omega-3 fatty acid other than the 15-HEPE comprise not more than about 10%, by weight, of all fatty acids present in the orally deliverable pharmaceutical composition.

8. The orally deliverable pharmaceutical composition of claim 7, wherein the omega-3 fatty acid is alpha linolenic acid, stearidonic acid, docosahexaenoic acid (DHA) or derivatives thereof.

9. The orally deliverable pharmaceutical composition of claim 1, comprising about 100 mg to 2000 mg of 15-HEPE in free form or a pharmaceutically acceptable ester, conjugate, or salt thereof.

10. The orally deliverable pharmaceutical composition of claim 9, comprising about 500 mg or about 1000 mg of 15-HEPE in free form or a pharmaceutically acceptable ester, conjugate, or salt thereof.

11. An orally deliverable dosage unit comprising: a capsule and a fill comprising about 100 mg to 5000 mg of 15-hydroxyeicosapentaenoic acid (15-HEPE) in free form or a pharmaceutically acceptable ester, conjugate, or salt thereof enclosed in a capsule shell, wherein the 15-HEPE or pharmaceutically acceptable ester, conjugate or salt thereof is the sole active ingredient and comprises at least about 80%, by weight, of all fatty acids present the orally deliverable dosage unit.

12. The orally deliverable dosage unit of claim 11, wherein the 15-HEPE is in esterified form.

13. The orally deliverable dosage unit of claim 12, wherein the esterified form is a C1-C5 alkyl ester.

14. The orally deliverable dosage unit of claim 12, wherein the esterified form is an ethyl ester.

15. The orally deliverable dosage unit of claim 12, wherein the esterified form is a triglyceride.

16. The orally deliverable dosage unit of claim 11, wherein the 15-HEPE is in salt form.

17. The orally deliverable dosage unit of claim 11, wherein an omega-3 fatty acid other than the 15-HEPE comprise not more than about 10%, by weight, of all fatty acids present in the orally deliverable dosage unit.

18. The orally deliverable dosage unit of claim 17, wherein the omega-3 fatty acid is alpha linolenic acid, stearidonic acid, docosahexaenoic acid (DHA) or derivatives thereof.

19. The orally deliverable dosage unit of claim 11, comprising about 100 mg to 2000 mg of 15-HEPE in free form or a pharmaceutically acceptable ester, conjugate, or salt thereof.

20. The orally deliverable dosage unit of claim 19, comprising about 500 mg or about 1000 mg of 15-HEPE in free form or a pharmaceutically acceptable ester, conjugate, or salt thereof.
